(19) United States
(12) Patent Application Publication
(10) Pub. No.: US 2010/0145293 A1
(43) Pub. Date: Jun. 10, 2010

(54) Apparatus and System for Isolating a Fistula

(76) Inventor: Amy L. Verhaalen, Cedarburg, WI (US)

Correspondence Address: ABSOLUTE TECHNOLOGY LAW GROUP LLC, 135 W. WELLS ST., SUITE 518, MILWAUKEE, WI, 53203, US

(21) Appl. No.: 12/704,282

(22) Filed: Feb. 11, 2010

Related U.S. Application Data
(63) Continuation-in-part of application No. 11/831,526, filed on Jul. 31, 2007.

(51) Int. Cl. A61F 5/445

(52) U.S. Cl. 604/337

(57) ABSTRACT

The present invention is an apparatus and system of isolating a fistula (e.g., an enteric fistula or an ostomy stoma) from within a wound bed, for use in conjunction with a negative pressure, suction-based dressing system comprised of an isolation component and a porous dressing component with an aperture that corresponds to the size and shape of the isolation component. The isolation component further includes an opening which is centered surrounding a fistula, exposing the fistula. The inner wall of the isolation component is air and fluid impermeable. The isolation component is secured to the wound tissue surrounding the fistula with adhesive while a negative pressure dressing system surrounds the isolation component.

### CROSS-REFERENCE TO RELATED APPLICATIONS

This application is a continuation-in-part application that claims priority to U.S. Nonprovisional application Ser. No. 11/831,526 filed on Jul. 31, 2007.

### FIELD OF INVENTION

The present invention is an apparatus for use with a suction-based dressing system for fistula isolation so that that fistula effluent does not interfere with the operation of a suction-based dressing system.

### BRIEF DESCRIPTION OF THE DRAWINGS

FIG. 1a illustrates an exploded view of an exemplary embodiment of an isolation system for isolating a fistula within a body wound.

FIG. 1b illustrates an assembled view of an exemplary embodiment of an isolation apparatus for isolating a fistula within a body wound which is to be used in conjunction with a suction-based dressing system.

FIG. 1c illustrates a sectional view of an exemplary embodiment of an isolation apparatus for isolating a fistula within a body wound which is to be used in conjunction with a suction based dressing system.

FIG. 2 illustrates a sectional view of an exemplary embodiment of an isolation apparatus isolating a fistula within a body wound.

FIG. 3 illustrates an exemplary embodiment of a modular component isolation apparatus for isolating a fistula within a body wound, including porous dressing material.

FIG. 4a illustrates an exemplary embodiment of a modular component isolation apparatus for isolating a fistula within a body wound.

FIG. 4b illustrates an exemplary embodiment of an isolation system for isolating a fistula within a body wound.

### GLOSSARY

As used herein, the term “fistula” refers to an abnormal passage leading from an abscess or hollow organ to the surface of the body permitting passage of fluids and secretions to the outside of the body including, but not limited to, an enteric fistula. As used herein, the term “fistula” shall also include a stoma or ostomy.

As used herein, the term “enteric fistula” refers to an abnormal passage between any organ within the gastrointestinal system and the surface of the body.

As used herein, the term “enteric contents” refers to bodily fluids and other substances which may be present in the intestines or within any organ of the gastrointestinal system.

As used herein, the term “stoma” refers to an artificial outlet from the gastrointestinal tract that permits drainage of human digestive components through the abdominal wall.

As used herein, the term “porous” refers to a component that is permeable to air, fluids or other molecules.

As used herein, the term “dressing” refers to a covering applied to a wound or the area surrounding the wound.

As used herein, the term “fluid impermeable” means resistant to the penetration of bodily fluids including, but not limited to, gastrointestinal drainage.

As used herein, the term “fluid impermeable structure” or “isolation component” refers to a component for isolating a portion of a wound including, but not limited to, a fistula or stoma. An isolation component may be round, oval, angular, square, rectangular, irregular or any other shape capable of isolating excrement from a fistula within a wound.

As used herein, the term “Negative Pressure Wound Therapy” or “NPWT” refers to a therapeutic technique which uses a vacuum source to create low pressure in the area surrounding the wound to promote healing of the wound. Examples of a vacuum source include, but are not limited to, those known in the art.

As used herein, the term “ostomy adhesive” or “adhesive component” refers to a substance which allows a material to adhere to a moist wound.

As used herein, the term “ostomy appliance” refers to a component used to collect bodily fluids from a wound area.

As used herein, the term “ostomy wafer” refers to the flattened component of an ostomy appliance which adheres to the skin or isolation apparatus.

As used herein, the term “modular” refers to a system having pre-configured components which may be selected and constructed to form an apparatus having the desired specifications.

As used herein, the term “pre-configured” means previously cut or configured to desired specifications.

### BACKGROUND

A fistula is an abnormal passage leading from an abscess or hollow organ to the surface of the body permitting passage of fluids and secretions to the outside of the body. Common types of fistulas include enteric or intestinal fistulas, gastric fistulas, pancreatic fistulas, and colon fistulas.

For a fistula type wound, a suction-based dressing system known in the art generally consists of a porous base dressing applied to a wound bed, a method to deliver negative pressure to the wound site, and a covering drape to separate the wound from the outer atmosphere.

A suction-based dressing system provides the benefit of improving granulation of tissue, as well as minimizing dressing change frequency and increasing patient comfort and mobility.

However, a suction-based dressing system has limited functionality when enteric contents are present. For example, a suction-based dressing system does not optimally function with solid material such as stool beneath the dressing. While mild to moderate liquid enteric contents may be successfully managed with a suction-based wound care system, once the enteric contents become thicker or the liquid volume becomes too great, the suction-based dressing system becomes much less successful and many times unusable.

When enteric contents (e.g., stool or air) seep into a suction-based dressing system, the suction function is interrupted. When the suction is interrrupted, the NPWT system may fail. An alarm may sound or enteric contents may pool onto the wound bed and surrounding healthy tissues.

Negative pressure from the suction-based dressing system may cause the stool or other enteric contents to enter the collection appliance interfering with the ability of the appliance to function properly.

It is desirable to have an apparatus that can be used in conjunction with existing suction-based dressing systems known in the art and which may allow such systems to function optimally when gastrointestinal contents are present.

It is further desirable to have an apparatus and system that facilitate the removal of bodily fluids from areas such as surgical wounds.

It is further desirable to have a method and apparatus capable of redirecting flow of bodily fluids to reduce collection system failure and reroute fluids away from undesired areas.

### SUMMARY OF THE INVENTION

The present invention is an apparatus for isolating a fistula (e.g., an enteric fistula or an ostomy stoma) for use in conjunction with a suction-based dressing system comprised of an isolation component and a porous dressing component with an aperture that corresponds to the size and shape of the isolation component. The isolation component further includes an opening which is centered on a fistula exposing the fistula. The inner wall of the isolation component is fluid impermeable and the entire apparatus is secured to the wound tissue surrounding a fistula using an ostomy adhesive.

### DETAILED DESCRIPTION OF INVENTION

For the purpose of promoting an understanding of the present invention, references are made in the text to exemplary embodiments of an apparatus and method for isolating a fistula within a body wound from surrounding tissue, only some of which are described herein. It should be understood that no limitations on the scope of the invention are intended by describing these exemplary embodiments. One of ordinary skill in the art will readily appreciate that alternate but functionally equivalent sizes, materials and techniques may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

It should be understood that the drawings are not necessarily to scale; instead, emphasis has been placed upon illustrating the principles of the invention. In addition, in the embodiments depicted herein, like reference numerals in the various drawings refer to identical or near identical structural elements.

Moreover, the terms “substantially” or “approximately” as used herein may be applied to modify any quantitative representation that could permissibly vary without resulting in a change in the basic function to which it is related.

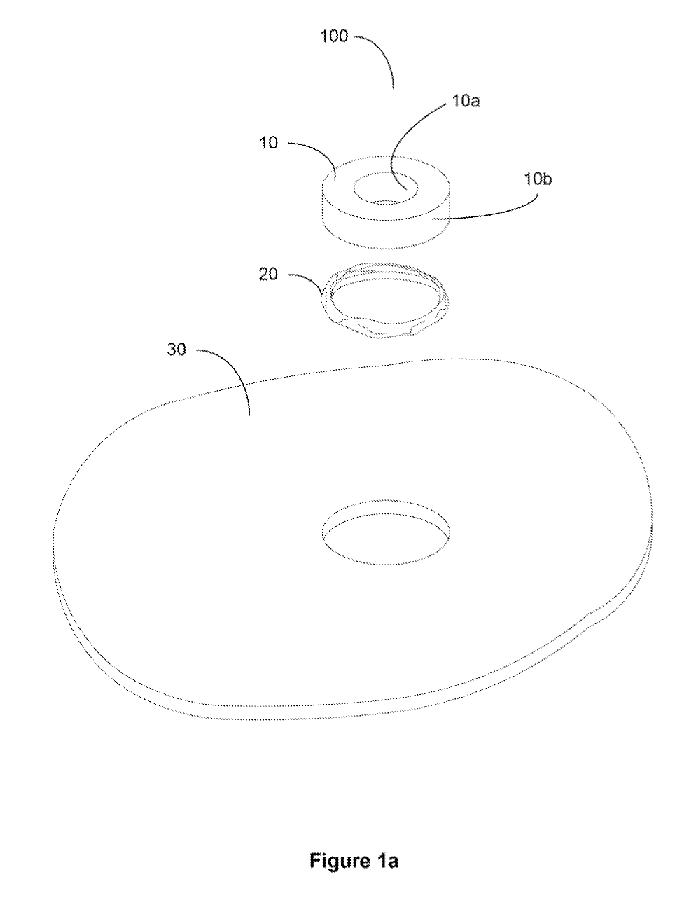

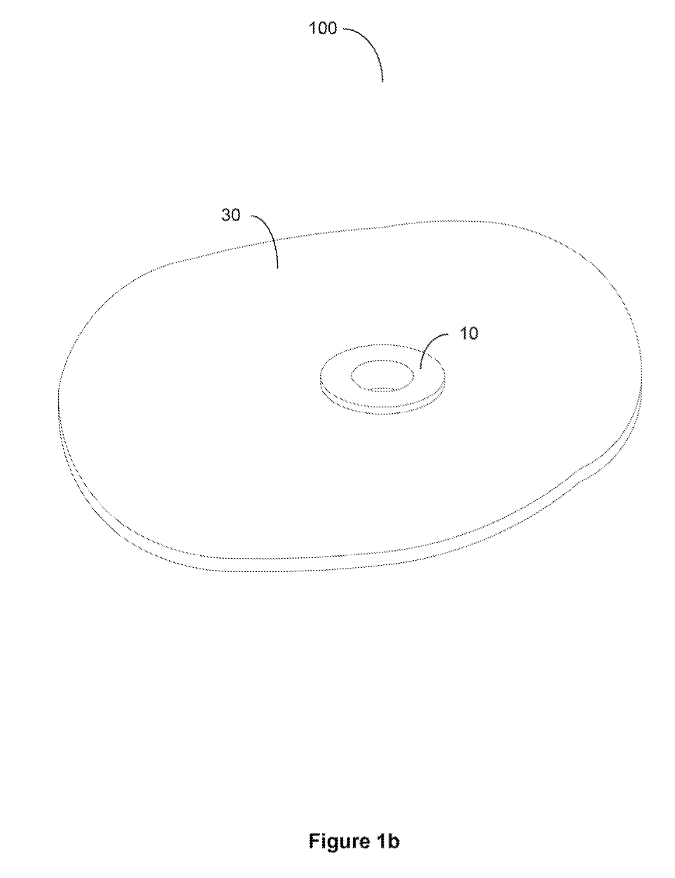

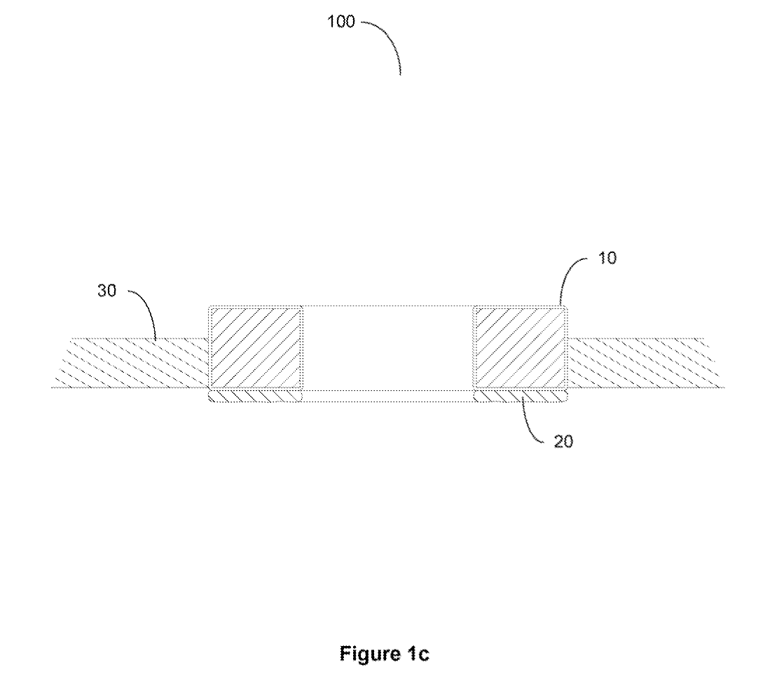

FIG. 1a shows an exploded view of an exemplary embodiment of isolation system 100 comprised of isolation component 10, adhesive 20 and porous dressing 30 having an aperture which surrounds the enteric fistula or ostomy stoma.

In the embodiment shown, isolation component 10 has a preformed opening which surrounds the enteric fistula or ostomy stoma. The dimensions of isolation component 10 will vary with the size of the wound. In various embodiments, isolation component 10 may have a height ranging from 0.5 to 1.5 inches, an outside diameter ranging from 1.5 to 4.0 inches, and a thickness ranging from 0.25 to 1.5 inches.

In the embodiment shown, isolation component 10 is comprised of foam and has inner wall 10a and outer wall 10b that are impermeable to both fluids and gases. In other embodiments, isolation component 10 may be comprised of plastic, coated gauze, nylon, rubber or any other medically safe material. In various embodiments, isolation component 10 may be disposable or non disposable.

In other embodiments, outer wall 10b of isolation component 10 may be either permeable or impermeable. When outer wall 10b is permeable it helps the suction-based dressing system maintain suction capability.

In still other embodiments, isolation component 10 may be comprised of multiple concentric rings of different materials having varying levels of permeability and/or may include various layers or coatings.

Fluid and other substances from the wound will drain into the opening of isolation component 10 and will remain inside isolation component 10 until removed. The fluid and other substances from the wound will not leak out and/or be soaked up by porous dressing 30 preventing the need for frequent dressing changes and decreasing the risk of skin irritation. The opening in isolation component 10 also provides access to the enteric fistula or ostomy for assessment or treatment.

In the embodiment shown, adhesive 20 is an ostomy adhesive known in the art.

In the embodiment shown, isolation component 10 has a preformed opening; however, in other embodiments, isolation component 10 may not have a preformed opening and an opening will need to be cut by a healthcare professional. In various embodiments, isolation component 10 may be constructed in various sizes to accommodate various size fistulas.

In the embodiment shown, porous dressing 30 is comprised of a flexible gauze material with pores that permit the passage of liquids and gases and provides uniform distribution of negative pressure when used with a suction-based dressing system for Negative Pressure Wound Therapy. The size and thickness of porous dressing 30 and the material from which porous dressing 30 is comprised varies with the size, type and location of the wound and the suction-based dressing system used. The size of the aperture in porous dressing 30 conforms to the perimeter of isolation component 10.

According to an aspect of the present invention, a method for isolating an enteric fistula or ostomy stoma from a contiguous body tissue area comprises the steps of adhesively applying an isolation component to a region of a body for which isolation of the region from surrounding tissue is desired and affixing a suction-based dressing to at least the surrounding tissue. The method also includes sealing the suction-based dressing to a wall of the isolation component and reducing pressure within the suction-based dressing to apply pressure to the surrounding tissue.

According to another aspect of the present invention, a method of tissue isolation comprises the steps of affixing an isolation component to a first tissue portion and attaching a low-pressure dressing to a wall of the isolation appliance and to a second tissue portion adjacent to the first tissue portion. A vacuum pressure is applied within the low-pressure dressing.

According to a further aspect of the present invention, an apparatus for isolating a fistula or ostomy stoma within a body wound includes at least one perimeter wall and a base attached to the at least one perimeter wall. An adhesive layer is attached to the base. The at least one perimeter wall and the base are formed of a foam that is impermeable to air and liquid.

FIG. 1b shows an assembled view of an exemplary embodiment of isolation system 100. Adhesive 20 is added to the bottom of isolation component 10 and isolation component 10 is placed inside the aperture in porous dressing 30. Porous dressing 30 and isolation component 10 are then placed over the fistula or ostomy stoma and surrounding area so that the aperture isolation component 10 surrounds the fistula or ostomy stoma creating an impermeable barrier between the fistula and porous dressing 30. Adhesive 20 (not visible) secures isolation component 10 to the area immediately surrounding the fistula.

In other embodiments, the bottom of isolation component 10 has a pre-applied adhesive covered by a paper backing which is removed before securing to the tissue surrounding the wound.

FIG. 1c shows the cross section of an exemplary embodiment of isolation system 100 with isolation component 10 secured inside the aperture of porous dressing 30.

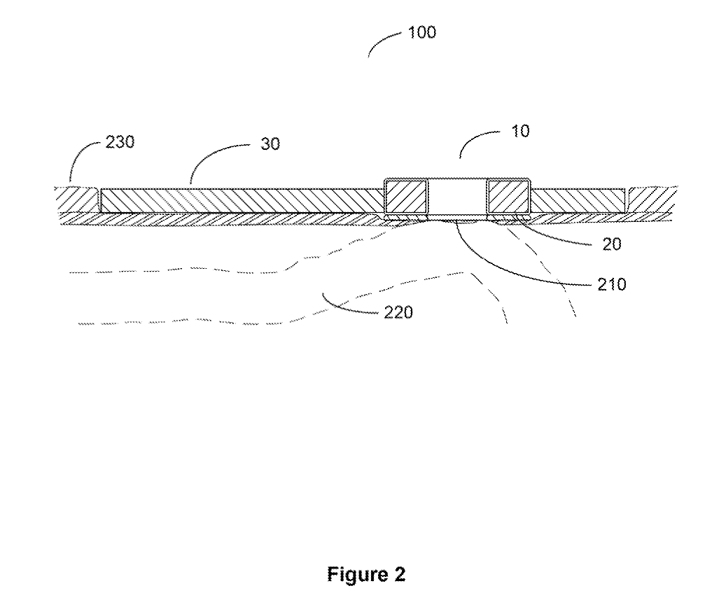

FIG. 2 shows the cross section of an exemplary embodiment of isolation system 100 in place over fistula 210. In the embodiment shown, fistula 210 is an enteric fistula between gastrointestinal component 220 and surface of the body. The center of isolation component 10 surrounds fistula 210 and porous dressing 30 rests against healthy tissue 230 surrounding fistula 210.

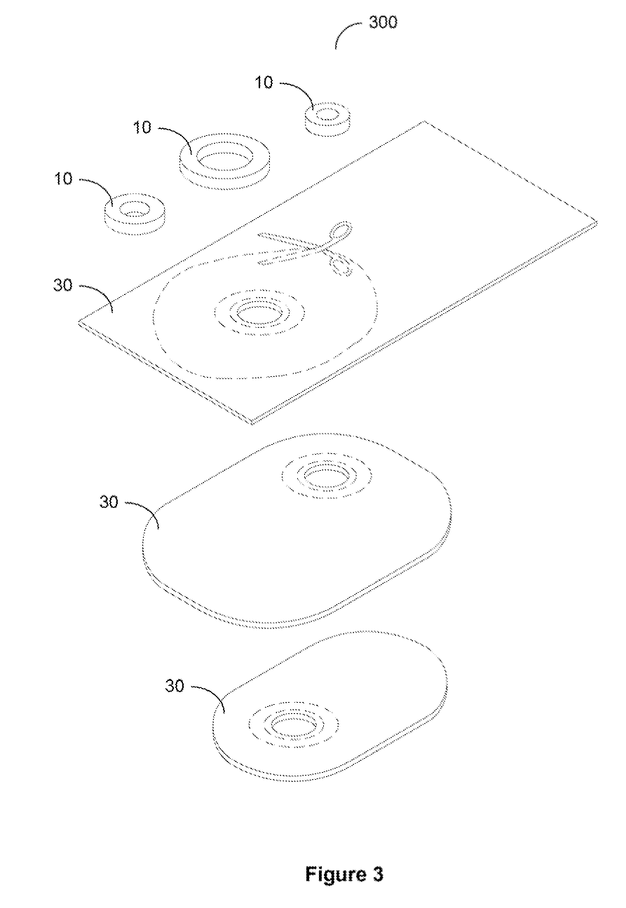

FIG. 3 shows an exemplary embodiment of modular component isolation apparatus 300 for isolation of a fistula or ostomy stoma within a body wound. In the embodiment shown, modular component isolation apparatus 300 is comprised of isolation components 10 of varying sizes and porous dressing 30. Porous dressing 30 is cut to the desired dimensions and an aperture which corresponds to the size of the desired size isolation component is cut in the desired location. FIG. 3 shows porous dressings 30 with various placements of the aperture.

In other embodiments, modular component isolation apparatus 300 may come with a smaller or greater number of isolation components and porous dressings and/or have porous dressings with pre-cut apertures. Modular component isolation apparatus 300 may also include dressings comprised of various materials. In still other embodiments, modular component isolation apparatus 300 may further include one or more covering drapes.

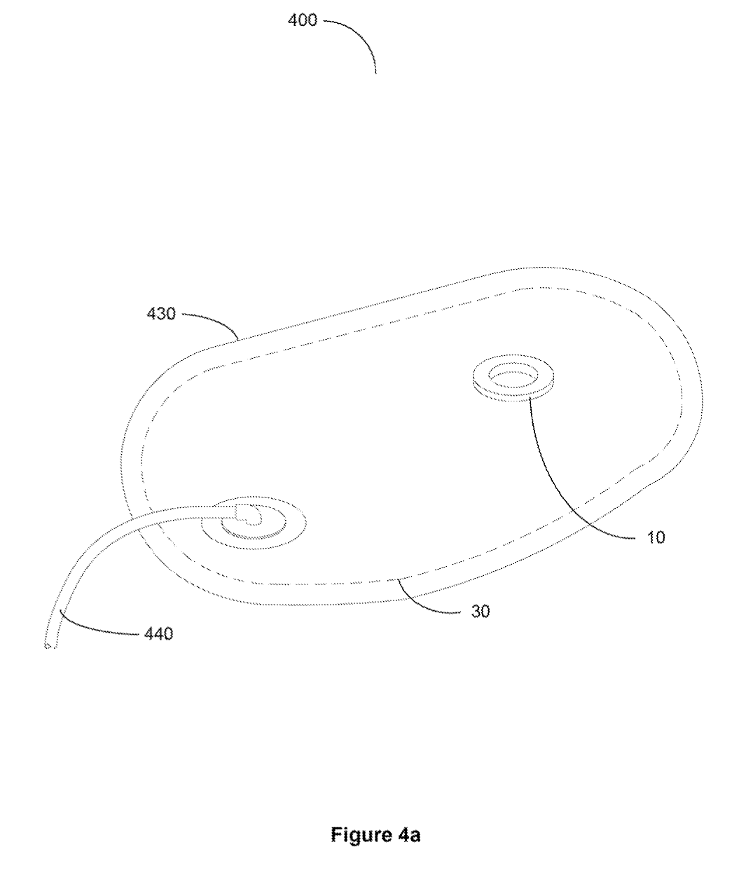

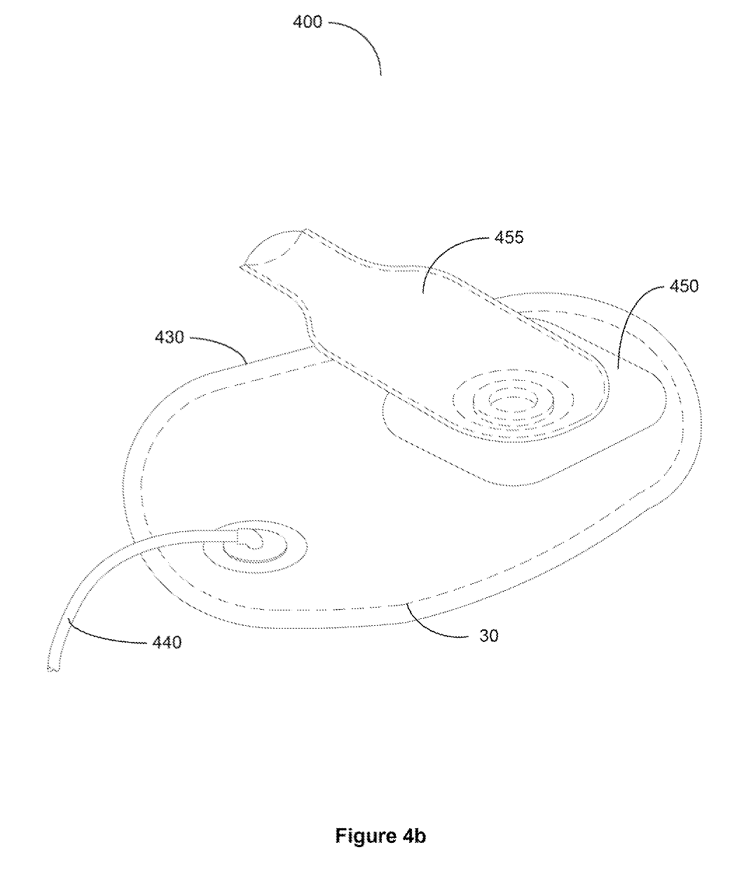

FIG. 4a shows an exemplary embodiment of isolation system 100 is use with suction-based dressing system 400. Isolation component 10 and porous dressing 30 are placed over the wound and surrounding area so that the center of isolation component 10 surrounds the fistula. Drape 430 having an opening which corresponds to the inside opening of isolation component 10 is placed over isolation component 10 and porous dressing 30 and secured to porous dressing 30 and surrounding tissue.

A suction-based dressing system is applied by forming a hole in drape 430 and placing suction catheter 440 over the hole. Suction catheter 440 removes air from the region covered by drape 430 lowering the air pressure. The air pressure inside the area surrounded by isolation component 10 is not affected by suction pressure and gastrointestinal fluids are not removed by the suction catheter, 440 preventing malfunction of suction catheter 440.

FIG. 4b shows an exemplary embodiment of isolation system 100 in use with use with suction-based dressing system 400 and stoma appliance 455. Stoma appliance 455 is secured to ostomy wafer 450 having an adhesive backing. An opening that is slightly larger than the size of isolation component 10 is cut into ostomy wafer 450. The paper is removed from the adhesive backing and the hole in ostomy wafer 450 is placed over the isolation component. Isolation component 10 creates an impermeable barrier around the fistula allowing suction-based dressing system 400 to function independently of ostomy appliance 455.

What is claimed is:

1. An apparatus for isolating a fistula from contiguous moist wound tissue area comprised of:
a porous dressing component having at least one aperture, said aperture adapted to provide access to said fistula, said aperture being further adapted to receive an isolation component, said isolation component having an impermeable inner surface, an outer surface, a top perimeter edge and a bottom perimeter edge.

2. The apparatus of claim 1 wherein said bottom perimeter edge is secured to the tissue surrounding said fistula with ostomy adhesive.

3. The apparatus of claim 1 wherein said isolation component is comprised of foam and further includes an adhesive component molded to form a fluid impermeable barrier between said surrounding wound and said bottom perimeter of said fluid impermeable component.

4. The apparatus of claim 1 wherein said isolation component is comprised of at least one inner layer of a first material and at least one outer layer of a second material.

5. The apparatus of claim 1 wherein said porous dressing component is gauze.

6. The apparatus of claim 1 wherein said porous dressing component is foam.

7. The apparatus of claim 1 wherein said adhesive component is a moldable adhesive component.

8. The apparatus of claim 1 wherein said aperture is cut to correspond to the size of said fistula.

9. The apparatus of claim 1 wherein said fistula is an enteric fistula.

10. The apparatus of claim 1 wherein said isolation component is a ring-shaped structure.

11. The apparatus of claim 1 wherein said isolation component has a height ranging from 0.5 to 1.5 inches, an outside diameter ranging from 1.5 to 4.0 inches, and a thickness ranging from 0.25 to 1.5 inches.

12. A modular system for constructing an apparatus for isolating a fistula from contiguous moist wound tissue area comprised of:
at least one porous dressing component constructed of a material which may be cut to the desired dimensions and which has an aperture in the desired location which corresponds to the size of an isolation component, said aperture adapted to receive and secure an isolation component; and
a plurality of isolation components, each having a pre-configured size and a pre-configured aperture.

13. The modular system of claim 12 wherein said at least one porous dressing component may be cut to a desired size and shape.

14. The modular system of claim 12 wherein one of said plurality of isolation components is selected, each of said isolation components having a specific size.

15. An apparatus for isolating enteric contents from a fistula comprised of:
a ring-shaped structure having an outer surface and an impermeable inner surface adapted to enclose said fistula and prevent said enteric contents from communicating with a porous dressing.

16. The apparatus of claim 15 wherein said outer surface is impermeable.

17. The apparatus of claim 15 wherein said outer surface is semi-permeable.

18. The apparatus of claim 15 wherein said outer surface is permeable.

19. The apparatus of claim 15 wherein said outer surface is textured.

20. The apparatus of claim 15 which further includes a drape, a porous dressing component, and an adhesive component.
